(19) United States
(12) Patent Application Publication
(10) Pub. No.: US 2026/0182824 A1
(43) Pub. Date: Jul. 2, 2026

(54) Heterogeneous Integration of Optical Elements into a Miniaturized Endoscopic System

(71) Applicant: Leadoptik Inc., San Jose, CA (US)

(72) Inventors: Mohammadreza Khorasaninejad, San Jose, CA (US); Soroosh Daqiqeh Rezaei, San Jose, CA (US); Jiang You, Fremont, CA (US); Xingdu Qiao, San Jose, CA (US); Thomas C. Pham, San Jose, CA (US)

(21) Appl. No.: 19/437,011

(22) Filed: Dec. 30, 2025

Related U.S. Application Data
(60) Provisional application No. 63/740,263, filed on Dec. 30, 2024.

(51) Int. Cl. A61B 1/00; A61B 1/06; A61B 1/07; A61M 25/00

(52) U.S. Cl. CPC A61B 1/00096; A61B 1/0623; A61B 1/07; A61M 2025/0042

(57) ABSTRACT

A technique is proposed for integrating optical components into a compact endoscopic system. In one instance, this involves combining a single/multimode fiber (or fiber bundle) and multiple metalenses using a ferrule and mounts. Specifically, the fiber is housed within the ferrule, which includes a protrusion that fits into a mount where the metalenses are securely positioned and aligned with the fiber. The integrated system allows light input through the fiber to interact with and be manipulated by the metalenses, enabling light delivery and focusing onto a target area. This configuration holds potential for miniaturized medical imaging across various modalities such as optical coherence tomography, fluorescence microscopy, and Raman spectroscopy.

### CROSS-REFERENCE TO RELATED APPLICATIONS

This application claims the benefit of U.S. Provisional Application No. 63/740,263, filed Dec. 30, 2024, which is incorporated in its entirety by this reference.

### BACKGROUND

### Field of Disclosure

This disclosure relates generally to endoscopes including optical systems, and, more particularly, miniaturization of optical systems in endoscopes using integrated lens mounts, self-alignment structures, and adhesive control.

### Description of the Related Art

Accurate diagnosis and treatment of diseases in internal organs, such as the pulmonary airways, coronary arteries, urological system, and gastrointestinal tracts, are challenging due to the difficulty in accessing lesions. This challenge drives the need for shrinking optical lighting and imaging technologies. However, miniaturizing medical lighting and imaging systems presents numerous obstacles. One significant challenge involves cascading multiple optical elements to create the necessary optical configuration for imaging.

As such, there is a need for technologies that integrate a cascading of miniaturized optical components into imaging systems, making them suitable for endoscopic applications. In particular, a system that combines a fiber with multiple metalenses using a ferrule and mounts. The alignment of the fiber and metalenses could be improved using a locking and self-aligning mechanism incorporated into the mount.

### SUMMARY

Disclosed herein are systems, methods, and apparatuses for heterogeneous integration of optical elements into a miniaturized endoscopic system. The system includes an integrated fiber-based illumination and imaging system that utilizes a unique mount structure to secure and align optical elements. The optical elements used to guide light may be diffractive, refractive, or metasurface-based lenses configured for the miniaturized endoscope. In some cases, a prism might also be used to guide light. The mount structure includes a series of self-alignment and locking features to facilitate the securing and aligning of the lenses (or other light guiding features). The system also implements several features designed to manage the application of glue during assembly. These features include microchannels for controlled glue dispensing and reservoirs for collecting excess glue. Finally, optical shielding has been implemented to safeguard the system from stray light, thus reducing noise and improving the performance of the system.

In some aspects, the techniques described herein relate to an endoscopic imaging system including: an optical fiber configured for translating light during imaging within a human body; a hypotube including: a proximal end and a distal end, wherein the optical fiber is coupled into the proximal end of the hypotube; and a window formed on an end wall at the distal end of the hypotube, the window including a structured slot on an outer perimeter of the end wall; and a lens configured to direct light to and from the optical fiber during imaging, the lens including a structured projection on an outer edge, the structured projection formed such that it passes through the structured slot in the end wall of the hypotube when mounting the lens in the hypotube.

In some aspects, the techniques described herein relate to an endoscopic imaging system, wherein the hypotube includes one or more vision windows, each of the one or more vision windows configured for transmitting light reflected off (or transmitted through) the lens or collecting light reflected from surfaces in the human body.

In some aspects, the techniques described herein relate to an endoscopic imaging system, wherein the window formed on the end wall is not one of the one or more vision windows.

In some aspects, the techniques described herein relate to an endoscopic imaging system, wherein the hypotube additionally includes: one or more attachment windows in a sidewall of the hypotube, the one or more attachment windows configured to secure the optical fiber into the hypotube, and wherein the optical fiber is secured into the hypotube by applying an adhesive substance to the one or more attachment windows.

In some aspects, the techniques described herein relate to an endoscopic imaging system, wherein: the lens is directly mounted to the hypotube by applying an adhesive substance to a distal side of the lens within the hypotube, and the adhesive substance is applied to the window on the end wall at the distal end of the hypotube and capillary forces draw the adhesive substance to the distal side of the lens.

In some aspects, the techniques described herein relate to an endoscopic imaging system, wherein the lens is mounted to the hypotube at a mounting angle relative to a light axis of the endoscopic imaging system, the mounting angle between a minimum angle and a maximum angle (e.g., between 0 and 180 degrees).

In some aspects, the techniques described herein relate to an endoscopic imaging system, wherein the minimum angle and the maximum angle is determined by absolute and relative positions of at least the lens, the optical fiber, and the hypotube.

In some aspects, the techniques described herein relate to an endoscopic imaging system, wherein the imaging system is configured to compensate for variations in the mounting angle between the minimum angle and the maximum angle.

In some aspects, the techniques described herein relate to an endoscopic imaging system, further including: a lens rest, wherein the lens is mounted on a surface of the lens rest within the hypotube, and wherein the lens reflects or transmits incoming light from the optical fiber.

In some aspects, the techniques described herein relate to an endoscopic imaging system, wherein: the lens rest is formed from a sidewall of the hypotube and is formed by bending the sidewall of the hypotube into a channel of the hypotube.

In some aspects, the techniques described herein relate to an endoscopic imaging system, wherein the lens rest includes: an insert for the hypotube, wherein the insert is configured for inserting into the distal end of the hypotube such that the lens is rested on a proximal surface of the insert.

In some aspects, the techniques described herein relate to an endoscopic imaging system, wherein the insert includes one or more channels, each of the one or more channels configured to receive an adhesive substance on a distal side of the insert to translate the adhesive substance to a proximal side of the insert.

In some aspects, the techniques described herein relate to an endoscopic imaging system, wherein the lens includes a proximal side and a distal side, the proximal side configured for reflecting or transmitting light in the endoscopic imaging system, and the distal side configured for resting the lens within the endoscopic imaging system.

In some aspects, the techniques described herein relate to an endoscopic imaging system, further including: a self-aligning lens mount, the self-aligning lens mount including one or more attachment structures configured to attach the lens to the self-aligning lens mount.

In some aspects, the techniques described herein relate to an endoscopic imaging system, wherein the one or more attachment structures are positioned such that the one or more attachment structures clamp the lens to the self-aligning lens mount along an outer perimeter of the lens.

In some aspects, the techniques described herein relate to an endoscopic imaging system, wherein the one or more attachment structures exert a spring force on a surface of the lens to couple the lens to the self-aligning mount.

In some aspects, the techniques described herein relate to an endoscopic imaging system, further including: a prism mount, wherein: the lens is coupled to the prism mount, and the prism mount directs light to and from the lens during imaging.

In some aspects, the techniques described herein relate to an endoscopic imaging system, wherein the prism mount directs light to and from the lens during imaging through a body of the prism mount.

In some aspects, the techniques described herein relate to an endoscopic imaging system, further including: an additional lens configured to direct light to and from the optical fiber during imaging, wherein: the additional lens is coupled to the prism mount, and the prism mount directs light to and from the lens during imaging.

In some aspects, the techniques described herein relate to an endoscopic imaging system, further including: one or more adhesive substance channels, each of the one or more adhesive substance channels formed to translate an adhesive substance from a first area of the endoscopic imaging system to a second area of the endoscopic imaging system, wherein capillary forces translate the adhesive substance from the first area to the second area through the one or more adhesive substance channels. [To be updated when claims are finalized.]

### BRIEF DESCRIPTION OF DRAWINGS

FIG. 1A illustrates a schematic of the integrated fiber-based illumination and imaging system, according to an example embodiment.

FIG. 1B shows a fiber with a core facet, according to an example embodiment.

FIG. 1C shows a ferrule with a channel that fiber passes through and a protrusion for inserting into a mount, according to an example embodiment.

FIG. 1D shows a mount for securing the ferrule and lenses in the system, according to an example embodiment.

FIG. 1E shows lenses for the lens mount, according to an example embodiment.

FIG. 2 shows a mount configured to self-align and lock the lens in a lens opening, according to an example embodiment.

FIG. 3 shows a mount configured to self-align and lock the lens in a lens opening, according to an example embodiment.

FIG. 4 shows a mount configured to self-align and lock the lens in a lens opening, according to an example embodiment.

FIG. 5 shows a small form factor endoscopic fiber-based imaging and illuminating system that stacks multiple lenses with self-aligning and self-locking features to enable multiple modalities, according to an example embodiment.

FIG. 6A shows a prism as a light routing element for the system and a lens mount for the prism, according to an example embodiment.

FIG. 6B shows a detailed cross-section view of the system with a prism for guiding light, according to an example embodiment.

FIG. 6C shows a detailed cross-section view of the system with a prism for guiding light, according to an example embodiment.

FIG. 6D shows a detailed cross-section view of the system with a prism for guiding light, according to an example embodiment.

FIG. 7A shows an end cut on the hypotube to facilitate the metalens mounting, according to an example embodiment.

FIG. 7B shows a lens rest integrated into the hypotube to securely hold the metalens, according to an example embodiment.

FIG. 8 shows a hypotube lens mount configured to facilitate rotation, according to an example embodiment.

FIG. 9 shows a hypotube lens mount configured to facilitate rotation, according to an example embodiment.

FIG. 10 shows a mount with microchannels for glue dispensing, according to an example embodiment.

FIG. 11 shows a mount with a glue collecting reservoir, according to an example embodiment.

FIG. 12 shows a mount with a glue collecting reservoir, according to an example embodiment.

FIG. 13 shows a mount with optical shielding, according to an example embodiment.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### Introduction

Conventional methods for fabricating miniaturized endoscopic imaging systems present significant technical and manufacturing challenges. Traditional assembly approaches typically require multiple discrete components (e.g., multiple separate ferrules, mounts, and alignment stages) to position optical elements relative to an optical fiber. Each component must be individually fabricated, aligned with sub-micrometer precision, and bonded in place, often requiring specialized alignment equipment and skilled manual labor. This multi-step process increases manufacturing complexity, extends production time, and introduces cumulative tolerances that can degrade optical performance. The need for high-precision active alignment during assembly is particularly problematic in high-volume production environments, where throughput and consistency are critical. Furthermore, the use of multiple components increases the overall size of the distal end of the endoscopic device, which can limit maneuverability within constrained anatomical spaces and reduce the range of accessible targets during minimally invasive procedures.

As an example, the integration of a hypotube as both a lens mount and fiber holder represents a departure from these conventional multi-component assembly approaches. The hypotube configuration disclosed herein consolidates the functions of ferrule, mount, and alignment stage into a single structural element, thereby reducing the number of discrete components and simplifying the assembly process. This consolidation addresses technical challenges associated with miniaturization, where space constraints and alignment tolerances become increasingly critical.

The structured slot and structured neck interface between the hypotube end wall and the lens provides a mechanical keying feature that facilitates repeatable angular positioning during assembly. Unlike conventional press-fit or adhesive-only mounting approaches, the slot-neck interface establishes a defined rotational orientation between the lens and the hypotube prior to final bonding. Moreover, it aids in establishing a mounting angle for the lens within the hypotube. Overall, this pre-alignment reduces the need for active alignment equipment during manufacturing, which can be particularly advantageous in high-volume production environments where throughput and consistency are important considerations.

Additionally, positioning of the lens mounting interface at the distal end wall of the hypotube, rather than along a sidewall, enables the lens optical axis to be oriented at a controlled angle relative to the fiber optical axis. This angular relationship can be designed to direct light toward specific anatomical targets during endoscopic procedures. The end wall mounting configuration also allows the lens to be positioned closer to the distal tip of the device, which can reduce the overall length of the imaging assembly and improve maneuverability within constrained luminal spaces.

In some example configurations, the hypotube may include one or more vision windows formed in the sidewall, distinct from the assembly window at the distal end. These vision windows permit light to exit or enter the hypotube for imaging purposes, while the assembly window serves primarily as a mechanical interface for lens installation. The separation of assembly and optical functions into distinct apertures allows each feature to be optimized independently. For example, the vision windows can be sized and positioned to maximize optical throughput, while the assembly window can be configured to provide mechanical stability and ease of manufacturing. If the assembly window and vision windows were not separated, the assembly window would need to serve dual purposes, which could compromise optical performance. Specifically, adhesive residue, mechanical stress concentrations, or dimensional tolerances required for lens mounting could obstruct or degrade the optical path, reducing image quality and introducing artifacts. Additionally, the structural modifications required for lens installation, such as the structured slot, could create optical discontinuities or scatter light, further degrading imaging performance. By maintaining separate apertures for assembly and vision functions, the system avoids these compromises and enables independent optimization of mechanical robustness and optical clarity.

The interplay between the structured neck and the structured slot is also beneficial. Indeed, the structured neck on the lens can be formed as an integral part of the lens substrate during fabrication, or it can be added as a separate feature through subsequent processing steps. The neck geometry can be tailored to match the slot geometry in the hypotube, with tolerances selected to provide a slip fit, transition fit, or interference fit as desired. In some cases, the neck may include chamfers or lead-in features to facilitate insertion during assembly, reducing the risk of damage to the lens or hypotube during handling.

Finally, the use of capillary forces to distribute adhesive from the assembly window to the lens mounting interface can improve bond uniformity and reduce the occurrence of voids or excess adhesive that might interfere with optical performance. By applying adhesive to the distal side of the assembly window and allowing it to wick toward the lens through controlled gaps or channels, the assembly process can be made more repeatable and less sensitive to operator skill. This approach can be particularly beneficial in miniaturized devices where manual adhesive dispensing is challenging due to the small feature sizes involved.

Beyond the hypotube-based configurations described above, the present disclosure encompasses additional embodiments that provide distinct advantages over conventional approaches in the field of miniaturized endoscopic imaging systems. These alternative embodiments address various technical challenges associated with optical alignment, component integration, and manufacturing efficiency.

One significant advantage shared across multiple embodiments is the incorporation of mechanisms that enable optical alignment within the endoscopic tool itself, thereby minimizing production steps and reducing potential sources of error. By integrating self-alignment features directly into the structural components of the endoscopic system, the embodiments disclosed herein reduce reliance on external alignment equipment and improve manufacturing repeatability.

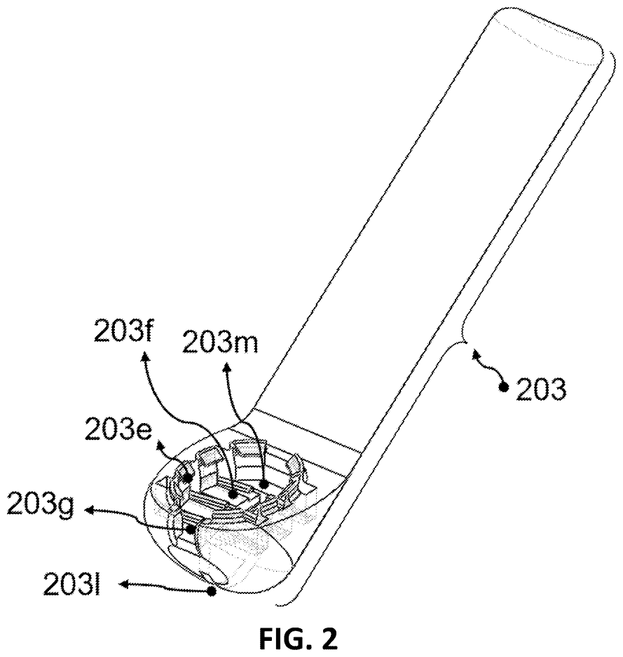

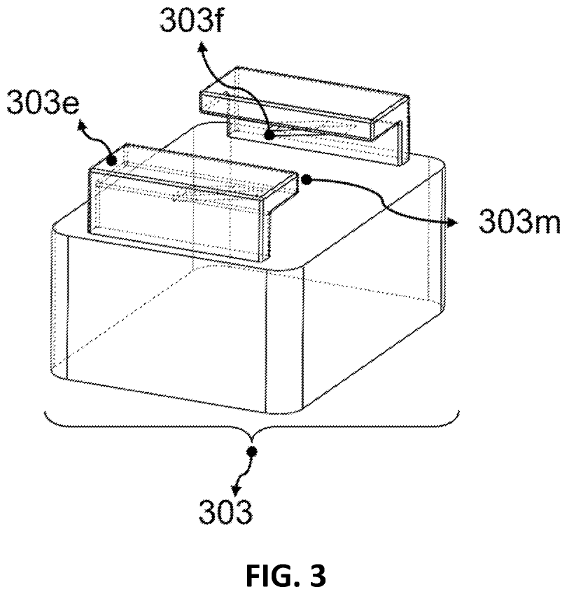

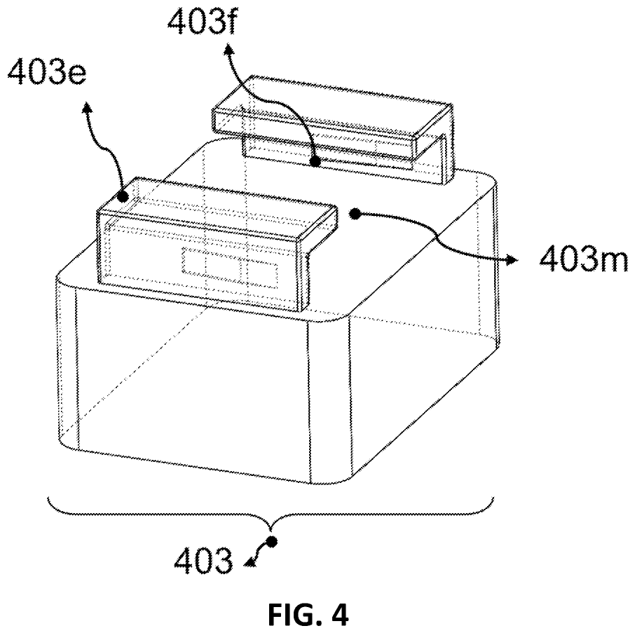

For example, the mount depicted in FIG. 2 is designed for fiber integration with minimal high-precision alignment required, featuring self-guiding and self-locking mechanisms. Similarly, the lens mount shown in FIG. 3A features self-aligning and self-locking capabilities where the lens is inserted into a slit and clamped and guided by integrated features, minimizing the use of glue as well as high-precision stages for alignments. FIG. 4 shows a mount that can integrate a fiber or ferrule with multiple lenses, where metalenses are fixed by self-locking features, with channels that guide the rays and limit noise level.

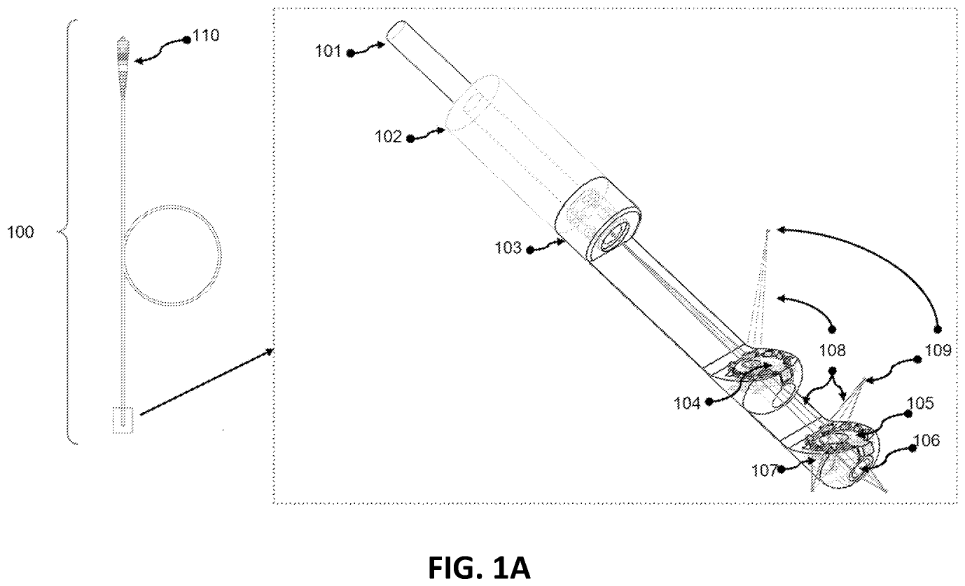

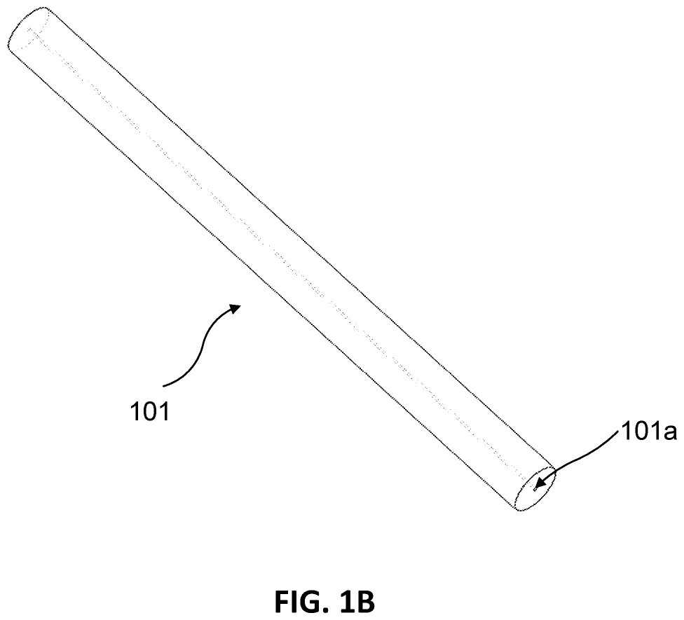

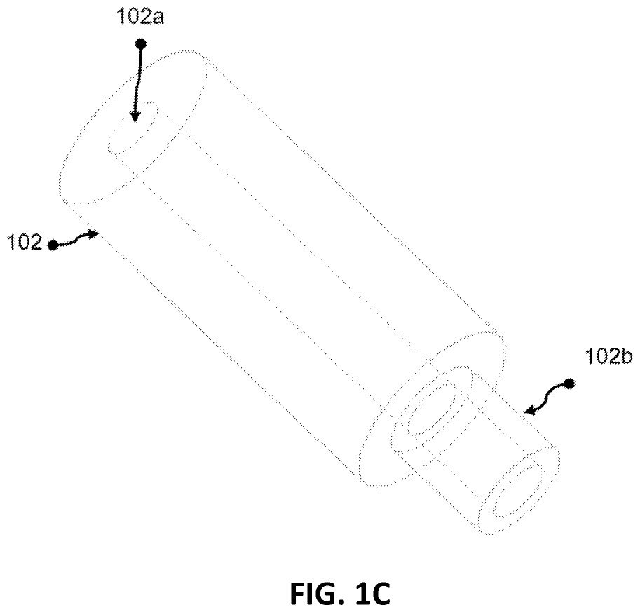

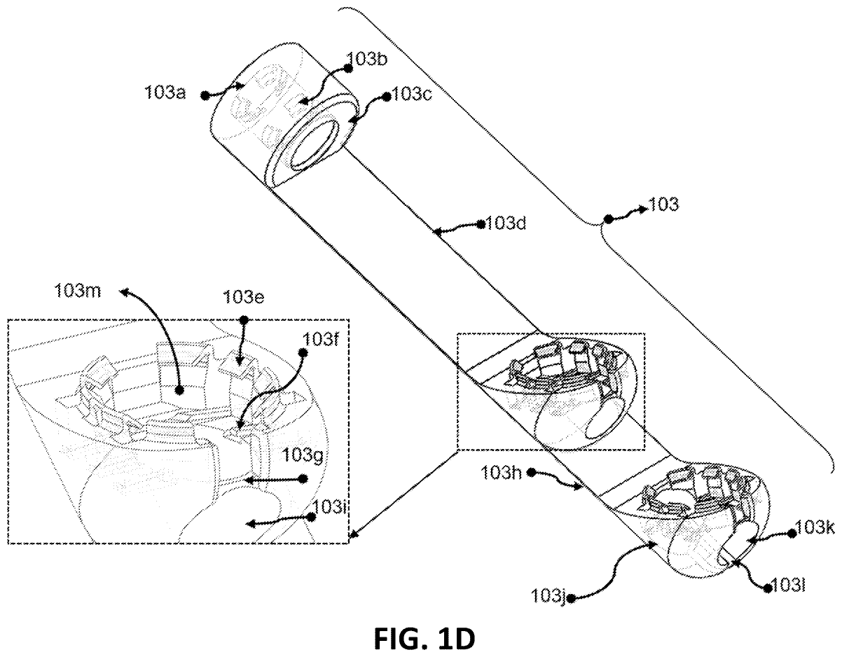

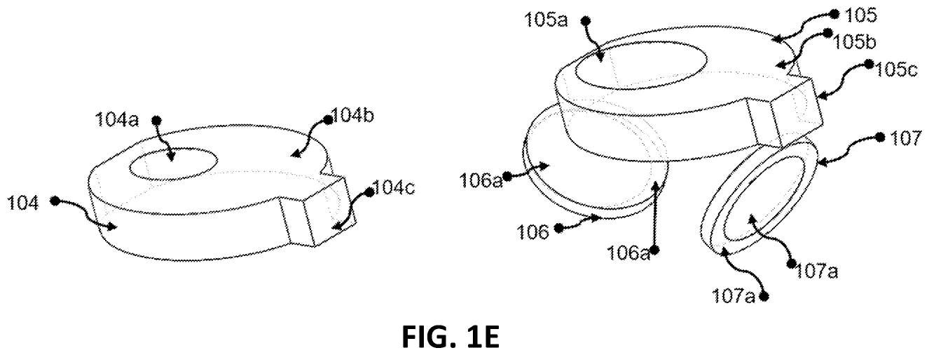

Ferrule-based approaches offer additional advantages in certain applications. The ferrule depicted in FIG. 1B includes a channel through which the fiber passes and a protrusion designed to fit into the mount, and the ferrule is used to facilitate handling of the fiber during the polishing process, which is crucial for controlling the light emission angle from the fiber and ensuring optimal performance of the miniature imaging system. The prism-based embodiments disclosed herein offer yet another set of advantages. An alternative approach involves using a prism, which inherently splits beams, to achieve the cascading effect, where the prism serves as the lens mount and light emitted from the fiber passes through the prism and strikes the rear interface of the metalens.

### Endoscopy Imaging System

FIG. 1A illustrates a schematic of the integrated fiber-based illumination and imaging system, according to an example embodiment.

The integrated fiber-based illumination and imaging system provides a compact optical assembly for directing and focusing light during endoscopic procedures. The system includes a fiber connector 110, an optical fiber 101, a ferrule 102, a mount 103, lenses 104-107, light rays 108, and focal points 109. The imaging system in FIG. 1A may include additional or fewer elements. Additionally, the elements depicted in FIG. 1A may have functionality different than what is described herein, or the functionality of one element may be attributable to a different element. Moreover, depending on the configuration, some of the functionality provided by depicted elements may be provided by elements external to the elements depicted in FIG. 1A.

The imaging system 100 (“system” hereinafter) includes an optical fiber 101. The optical fiber 101 is configured for translating light during imaging within a human body. The fiber 101 can be single mode or multimode fiber 101 but is not limited to these two types. The working wavelength of the device can range from UV to Visible to Infrared regions of the electromagnetic spectrum. The fiber length can range from a few centimeters to a couple of meters.

As illustrated, the system 100 includes a ferrule 102. The ferrule 102 provides a mechanical housing for the optical fiber 101 and facilitates optical connectivity by enabling precise alignment of the fiber 101 with downstream optical components. The ferrule 102 diameter can vary from 10 μm to 5 mm with typical size of 300 μm. The ferrule 102 length can range from 100 μm to 10 mm with typical size of 1.5 mm.

The system 100 includes a mount 103. The mount 103 provides a structural framework for maintaining optical connectivity between the ferrule 102 and the lenses 104-107 by securing these components in fixed spatial relationships. The ferrule 102 may be inserted into the mount 103. Lenses 104-107 are secured and aligned within the mount 103. The mount 103 diameter can vary from 10 μm to 5 mm with typical size of 300 μm. The mount 103 length can range from 100 μm to 10 mm with typical size of 2 mm.

The system 100 includes one or more lenses (e.g., lenses 104-107). The lenses 104-107 are configured to direct and focus light rays 108 emitted from the optical fiber 101 toward target areas within the human body. The lenses 104-107 interact with the optical fiber 101 and the mount 103 to form an integrated optical path for imaging and illumination. The lenses 104-107 may be diffractive, refractive, metasurface-based, or a combination of all. The mode principle of lenses can involve transmission mode, reflection mode, or both. The lenses 104-107 thickness can range from 1 μm to 2 mm (typical 50 μm) with footprint of 100 μm to 20 mm (typical 250 μm).

The system 100 in aggregate forms an optical path for the imaging system. For instance, light rays 108 emitted from the fiber 101 are directed and focused at focal point 109 by the lenses. Focal points 109 can be target areas for illumination and/or imaging. The focal point 109 may be external to the system 100, such as when the system 100 is used for imaging inside a human body.

The imaging system 100 includes a standard fiber connector 110. The fiber connector 110 provides a standardized interface for coupling the optical fiber 101 to external light sources or detection systems. As illustrated, the fiber connector 110 is an SC connector, but other fiber connectors are also possible. For instance, the fiber connector 110 may be an SC/APC, FC, FC/APD, LC, etc. fiber connector.

FIG. 1B shows a fiber with a core facet, according to an example embodiment. The fiber 101 is configured for translating light during imaging within a human body. Within the fiber 101, the core facet 101a is the part of the fiber where light travels. The core facet 101a may be polished to control the light emission angle from the fiber, allowing improved performance of the imaging system. The fiber 101 coating may be stripped or reduced before inserting into the ferrule channel to facilitate proper optical coupling.

FIG. 1C shows a ferrule with a channel that fiber passes through and a protrusion for inserting the ferrule into a mount, according to an example embodiment. The fiber 101 of FIG. 1B may pass through the channel in the ferrule 102. The channel is an opening extending from a first side 102a to a second side 102b of the ferrule 102. The second side 102b of the ferule 102 is configured to be coupled to the mount 103. The fiber 101 coating may be stripped or reduced before inserting into the ferule channel on the first side 102a. The ferrule 102 may function to polish the fiber 101, which can be useful in increasing performance of the system 100.

FIG. 1D shows a mount in the system, according to an example embodiment. The mount 103 secures the ferrule 102 and lenses 104-107 within the system 100. The mount 130, due to its configuration, may cause alignment free positioning and stacking of the lenses. The illustrated mount 103 is constructed with features that reduce the work typically necessary to align optical elements. The mount 103 is also constructed in a manner that reduces the need for adhesive substances (e.g., glue).

The mount 103 has a channel through its center. The channel joins a first side 103a of the mount to a second side 103c of the mount. The second side 102b of the ferrule is reciprocally couplable to the first side 103a of the mount 103. The mount channel also includes alignment elements 103b. The alignment elements enable the ferrule 102, when inserted into the first side 103a of the mount using its second side 102b, to self-align. The self-alignment may be caused by, e.g., the locking elements 103b affixing to the ferrule 102 and aligning mount 103.

The mount 103 includes a stopper wall 103c to stop the ferrule 103. The stopper wall 103c may be the second side 103c of the mount channel. The stopper wall 103c prevents the ferrule 102 from being over inserted into the mount 103. The mount 103 includes a bridge 103d. The bridge 103d provides a structural connection between the mount 103 and lenses 104-107, and also a bridge 103h between lens mounts.

The lens mounts (enlarged inset) include a lens opening e.g., 103m. The lens opening 103m also includes self-aligning and locking elements 103e to secure and align a lens (e.g., lens 104). That is, the self-aligning elements 103e are reciprocally couplable to the lens that rests in the lens mount.

In some example configurations, springs 103f keep the lens 104 within a certain distance from fiber 101. The lens mount includes a slot 103g for a lens neck 104c (not shown). The lens slot 103g is configured such that the lens 104 may be placed into the lens neck 104c for alignment and easy breakage and what would be connected to lens 104 for carrying it. The lens slot 103g is configured to accommodate the lens neck 104c (not shown). The lens neck 104c connects the lens 104 to a carrier body which is generally larger than the lens for easy handling. When the lens 104 is secured into the lens opening 103m, the lens will be pressed against the lens opening 103m which will result in the lens neck 104c breakage. The lens slot 103g facilitates the lens neck 104c to break easily from what would be connected to lens 104 for carrying it.

The lens mount includes an opening 103i. The lens opening 103i allows rays from the fiber 101 to pass through. The lens mount also includes an opening 103j on the back to place another lens 106 (not shown). The lens mount also includes an opening on the front 103k. Another lens 107 (not shown) may be placed on the front opening 103k. The lens mount may also include a trench 103l. The trench is configured to allow glue dispersion through the trench, which allows the glue to bond and fix the lenses 106-107 in the mount.

FIG. 1E shows a lens for a lens mount, according to an example embodiment. The illustrated lenses may be any of lenses 104-107 utilized in this imaging system. Lens 104 illustrated in reflection mode with beam-splitting capabilities. The beam-splitting/focusing area is at 104a, and the substrate is 104b. Half of the light incident on the beam-splitting area 104a is reflected and focused on a target area, while the remaining half is transmitted through the substrate 104b to illuminate the subsequent lenses 105-107. Lens 104 may be a metalens (metasurface-based lens) or a diffractive lens. The lens neck 104c is the part left after the first lens 104 is separated from what it is originally connected to for handling it.

Similarly, lens 105 is reflective metalens with a beam-splitting section 105a, a supporting substrate 105b, and a neck 105c. The beam-splitting area 105a directs half of the incoming light to the third metalens 106 and transmits the other half to the fourth metalens 107. The ratio at which the beam splitting divides the light can be 50:50, 40:60, 30:70, 60:40, 70:30, or any arbitrary ratio. Beam splitting might introduce some loss while splitting the incoming light. Both metalenses 106 and 107 feature a transmissive lens area (106a and 107a, respectively) that focuses incoming light on a target area, along with a substrate (106b and 107b) to support the metalenses.

The lenses described above represent example configurations, and other lens types and operational modes are also possible within the scope of the present disclosure. For instance, the lenses may operate in transmission mode only, reflection mode only, or a combination of both transmission and reflection modes. The lenses may be configured as refractive lenses, diffractive lenses, metasurface-based lenses (metalenses), or hybrid combinations thereof. In some configurations, the lenses may be polarization-sensitive, wavelength-selective, or designed to manipulate the phase, amplitude, or angular momentum of incident light. Additionally, the lenses may be configured to focus light at a single focal point or at multiple focal points, enabling multi-plane imaging or depth-resolved imaging within the human body. The lenses may also be designed to operate across different wavelength ranges, including ultraviolet, visible, near-infrared, or mid-infrared regions of the electromagnetic spectrum, depending on the imaging modality and clinical application.

### Self-Alignment Systems

The self-alignment systems disclosed herein address the manufacturing challenges introduced in the preceding section by integrating mechanical features that enable passive optical alignment during assembly. Rather than relying solely on external alignment equipment or iterative adjustment procedures, these systems incorporate structural elements (e.g., such as slits, fins, and spring-loaded clamps) that guide optical components into their intended positions through mechanical interaction with the system 100 itself. This approach reduces the need for skilled manual intervention and high-precision stages, thereby improving manufacturing throughput and consistency.

The configurations presented in FIGS. 2 through 4 illustrate variations of this self-alignment principle, each employing a different mechanical strategy to secure and position lenses within a mount. In each case, the mount includes features that constrain the lens in multiple degrees of freedom, establishing both translational and rotational alignment relative to the optical fiber. By consolidating alignment functions into the mount geometry itself, these systems reduce the number of discrete assembly steps and minimize cumulative tolerances that can degrade optical performance in miniaturized endoscopic devices.

FIG. 2 shows a mount configured to self-align and lock the lens in a lens opening, according to an example embodiment. The mount may be the same mount, or a portion of the mount discussed in FIGS. 1A-1E. The mount 203 is configured to self-align and lock the lens in lens opening 203m. The mount locks the lens into the lens opening using fins 203e. The fins 203e align and lock the lens in place when the lens is inserted into the lens opening. To do so, the springs 203f backload the lens (e.g., contact the back of the lens) and keep the position of lens within a specific location in case the thickness of lens had deviations from designed value (e.g., by exerting a spring force to press and hold the lens into place). In some configurations, the mount 203 may include a lens neck slot 203g for lens neck breakage, and trench 203l for extra glue to flow out of the lens opening 203m.

FIG. 3 shows a mount configured to self-align and lock the lens in a lens opening, according to an example embodiment. The example in FIG. 3 is different from FIG. 2 in that it leverages a top-loaded spring-lock configuration. To expand, lens mount 303, again, includes mechanisms for self-aligning and self-locking a lens in the lens mount. To do so, the mount 203 is spring-loaded from top (rather than the back). In this example, a lens is inserted into the lens opening 303m. Slits 303e on the mount guide the lens into position, and the lens is clamped by springs 303f from the top. This approach reduces the use of glue as and the need for high-precision stages for alignments.

FIG. 4 shows a mount configured to self-align and lock the lens in a lens opening, according to an example embodiment. The example in FIG. 4 is similar to FIG. 3, but leverages a side-loaded spring-lock configuration. To expand, lens mount 303, again, includes mechanisms for self-aligning and self-locking a lens in the lens mount. To do so, the mount 403 is spring-loaded from side (rather than the top or back). In this example, a lens is inserted into the lens opening 403m. Slits 403e on the mount 303 guides the lens into position, and the lens is clamped by springs 403 from the side.

The self-alignment configurations described above represent example mechanical approaches, and other mechanical self-alignment modes are also possible within the scope of the present disclosure. For instance, alternative configurations may employ ball-and-socket joints to provide rotational alignment while constraining translational degrees of freedom, or may utilize tapered interfaces that guide components into alignment through wedge-shaped features. Other configurations may use flexure-based mechanisms that provide compliant alignment through elastic deformation of structural elements, or may employ kinematic coupling principles using three-point contact geometries to establish repeatable positioning. Additionally, some mounts may incorporate threaded interfaces that convert rotational motion into controlled axial positioning of optical elements.

The mechanical self-alignment techniques disclosed herein for lens positioning may be applied to other elements of the endoscopic imaging system. For example, similar self-aligning and self-locking features may be incorporated into ferrule mounts to facilitate passive alignment of the optical fiber within the system, reducing the need for active alignment equipment during fiber installation. The prism-based configurations may also benefit from self-alignment mechanisms that establish the angular relationship between the prism facets and the optical axis of the fiber. In hypotube-based assemblies, self-alignment features may be integrated into the hypotube end wall or sidewall to guide the lens into its intended position during insertion through the assembly window. Additionally, self-alignment principles may be extended to the integration of multiple optical elements in cascaded configurations, where each element includes mechanical features that establish its position relative to adjacent components. Such approaches can reduce cumulative alignment errors and improve manufacturing repeatability across the entire optical assembly.

### Multiple Modality Configurations

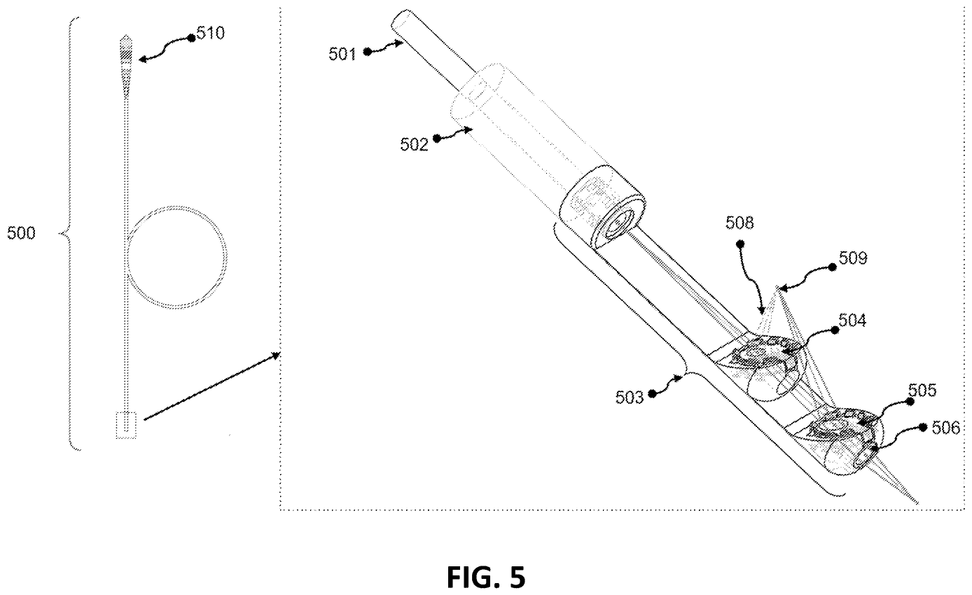

FIG. 5 shows a small form factor endoscopic fiber-based imaging and illuminating system that stacks multiple lenses with self-aligning and self-locking features to enable multiple modalities, according to an example embodiment. The different, stacked lenses allow the system to capture information in different modalities such as, e.g., wavelength, polarization, spin, and angular momentum.

The multi-modality configuration is similar to that shown in FIG. 1. That is, the integrated fiber-based illumination and imaging system 500 includes a standard connector (SC) 510. The optical fiber 501 is contained within a ferrule 502 that is inserted into a mount 503. Metalenses 504-507 are secured and aligned within the ferule. Light rays 508 emitted from the fiber are directed and focused 509 by the metalenses onto target areas for illumination and/or imaging. Light rays 508 reflected off lens 504 and 505 focuses on the same focal point 509 that facilitates gathering of various modal information from a single location. This configuration enables the acquisition of diverse data, including (but not limited to) field of view, depth of field, imaging resolution, etc., all from the same point.

### Optical Element Lens Mount

While the self-alignment systems described in the preceding sections provide certain advantages, other types of mounts are also possible within the scope of the present disclosure. These alternative mounting configurations may take forms distinct from those described above, and in some cases, the lens mounts themselves may provide additional optical functionality beyond mechanical support and alignment.

For instance, a lens mount may be configured to act as a beam splitter, a wavelength filter, a polarization controller, or a light-routing element that directs light along specific optical paths within the endoscopic imaging system. By integrating optical functionality directly into the mounting structure, such configurations can reduce the total number of discrete optical components required and simplify the overall assembly of the system.

To provide an example, in some configurations, a prism can act as light-routing elements in the system. The prism guides light toward a designated direction to reach a designed lens. For light-guiding mechanisms, prisms may rely on various effects, such as reflection, refraction, or total internal reflection. Additionally, certain facets of the prisms can be coated with thin films or a stack of thin-film layers to control reflection and transmission from those surfaces.

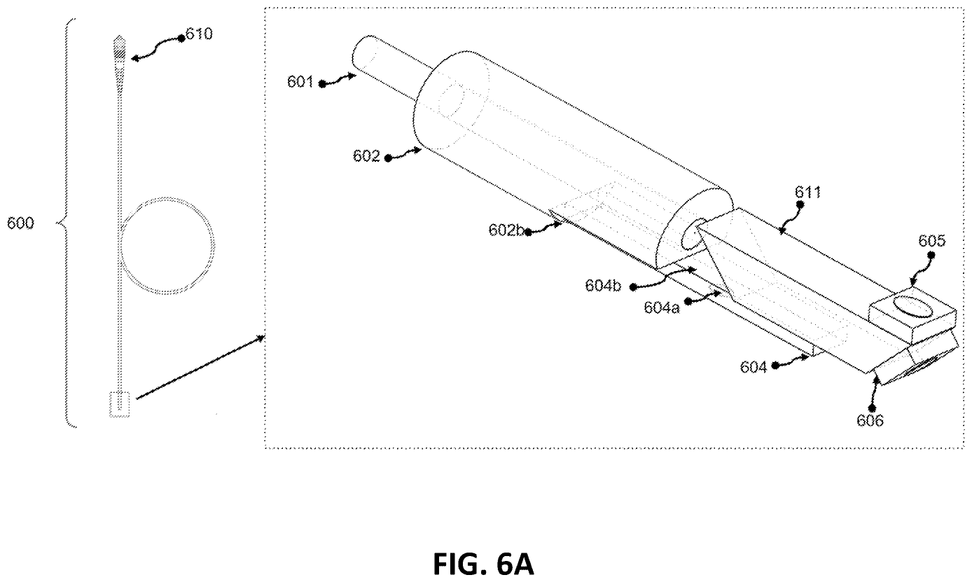

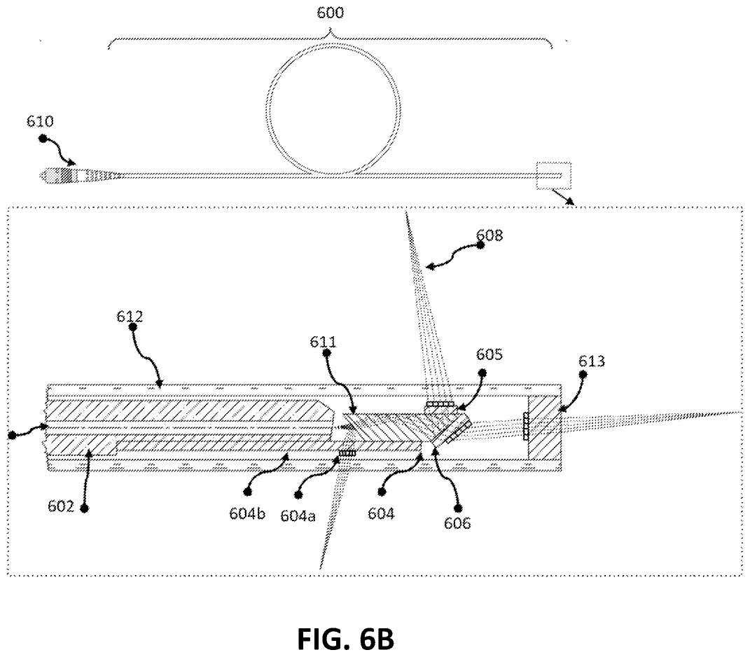

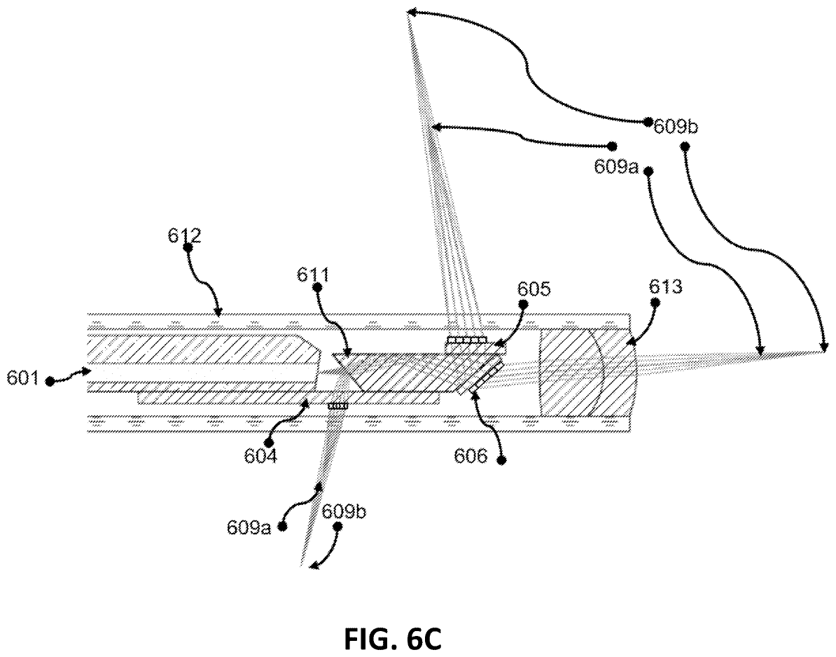

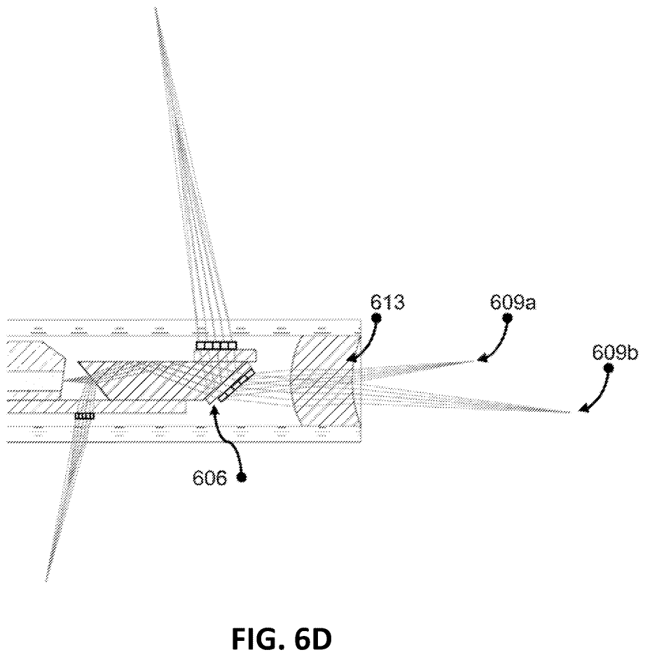

FIG. 6A shows a prism as a light routing element for the system and a lens mount for the prism, according to an example embodiment. The prism 611 serves as a light routing elements for the system 600.

Similar to the system 100 of FIG. 1, the system 600 includes a fiber 601, a ferrule 602 and its stopper 602b. The system also includes a bridge lens 604 with its focusing/lens region 604a and substrate 604b.

The system 600 differs from the ones described above. For instance, the system 600 includes a prism 611 that is utilized as the mounting structure for the lenses (e.g. metalenses). In this configuration, light emitted from the fiber first strikes the initial facet of the prism 611 (the obscured surface). A portion of this light is directed to lens 604, while the remaining light passes through the prism 611 toward the rear interface, where lens 606 is mounted. The rear interface of the prism 611 splits the light, directing half toward lens 605 and the other half to lens 606. The rear interface can adjust the light split ratio, allowing for distributions other than 50:50, such as 40:60, 30:70, 60:40, 70:30, or any other arbitrary ratios. In some configurations, an external tubing or sheath can be added to encapsulate the system 600 for additional protection. This sheath can also provide extra functionality by allowing the integration of more lenses and enhancing durability.

FIG. 6B shows a detailed cross-section view of the system with a prism for guiding light, according to an example embodiment. The system includes a fiber 601, ferrule 602, prism 611, and lenses 604, 605, 606 that, acting together, guide the light through the system 600. Elements of the system are protected by an outer sheath 612. The distal tip is capped by a forward viewing lens 613. The external tubing/sheath is to protect the probe as well as adding extra functionality by integrating more lenses.

FIG. 6C shows a detailed cross-section view of the system with a prism for guiding light, according to an example embodiment. The example of FIG. 6C also includes a fiber 601, ferrule 602, prism 611, lenses 604, 605, and 606, and forward-looking lens 613. The forward-looking lens can be a doublet lens to correct for aberrations. As shown here, the lenses 604, 605, 606 can have multiple focal points 614, 615, 616, 617 and the forward-looking lens 613 can be a doublet lens to correct for aberrations. Lenses 604, 605, and 606 may be polarization-dependent metasurface lenses that implement different phases depending on the impinging light. Therefore, the light will focus at different focal spots depending on its polarization. The polarization of light can be linear, circular, elliptical, or a combination of these. The lens 606 may act as a beam splitter as well as a lens.

FIG. 6D shows a detailed cross-section view of the system with a prism for guiding light, according to an example embodiment. The example of FIG. 6D also includes a fiber 601, ferrule 602, prism 611, lenses 604, 605, and 606, and forward-looking lens 613. As shown here, the metalens/beam splitter 606 can break the incoming light into two different diffraction orders depending on light polarization (it also can be based on wavelength that will be focused by a forward-looking lens 613. In this example, the forward-looking lens 613 is a concavo-convex lens focusing light at two different focal points 614, 615.

In some configurations, the prism 611 may be inserted into the outer sheath 612 from the distal end and subsequently capped to secure the assembly. For example, the prism 611 may be positioned within the outer sheath 612 such that its facets align with the optical axis of the fiber 601, and a distal cap or lens 613 may then be affixed to the distal end of the outer sheath 612 to enclose the prism 611. The prism 611 may be affixed within the outer sheath 612 by applying an adhesive substance to one or more slots or channels formed in the inner wall of the outer sheath 612, or by mechanical interference fit between the prism 611 and the inner diameter of the outer sheath 612. This end-insertion approach simplifies assembly by allowing the prism 611 to be positioned and secured in a single operation, reducing the number of alignment steps required during manufacturing. Additionally, one or more of the self-alignment structures described above may be applied to the prism.

Additionally, the prism-based configuration enables both sidewall and end wall viewing within the endoscopic imaging system. Light emitted from the fiber 601 may be directed by the prism 611 toward lenses 604 and 605 positioned on the sidewalls of the outer sheath 612, enabling lateral imaging of tissue structures adjacent to the endoscope. Simultaneously, light may be transmitted through the prism 611 to the forward-looking lens 613 at the distal end, enabling forward imaging along the longitudinal axis of the endoscope. This dual viewing capability allows the system 600 to acquire images from multiple perspectives without requiring repositioning of the endoscope, which can improve diagnostic accuracy and reduce procedural time. The prism 611 thus functions both as a structural mount for the lenses and as an optical element that enables multi-directional imaging within a compact form factor.

### Hypotube Lens Mount

The hypotube-based configurations discussed in this section address the manufacturing challenges introduced in the preceding sections by consolidating multiple assembly functions into a single structural element. As noted above, conventional methods for fabricating miniaturized endoscopic imaging systems typically require multiple discrete components, each of which must be individually fabricated, aligned with sub-micrometer precision, and bonded in place. This multi-step process increases manufacturing complexity, extends production time, and introduces cumulative tolerances that can degrade optical performance. The hypotube configuration consolidates the functions of ferrule, mount, and alignment stage into a single structural element, thereby reducing the number of discrete components and simplifying the assembly process.

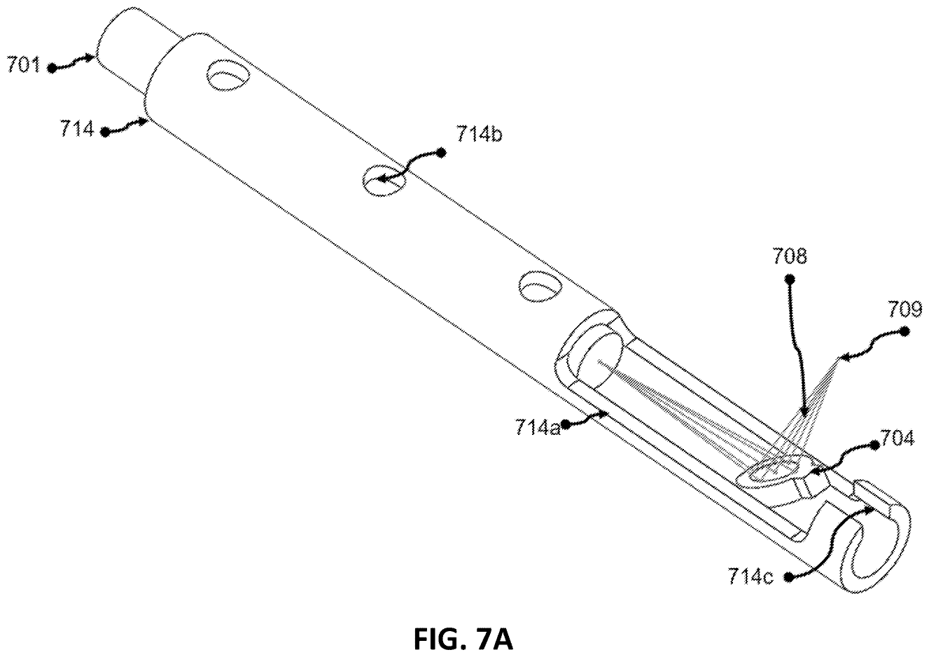

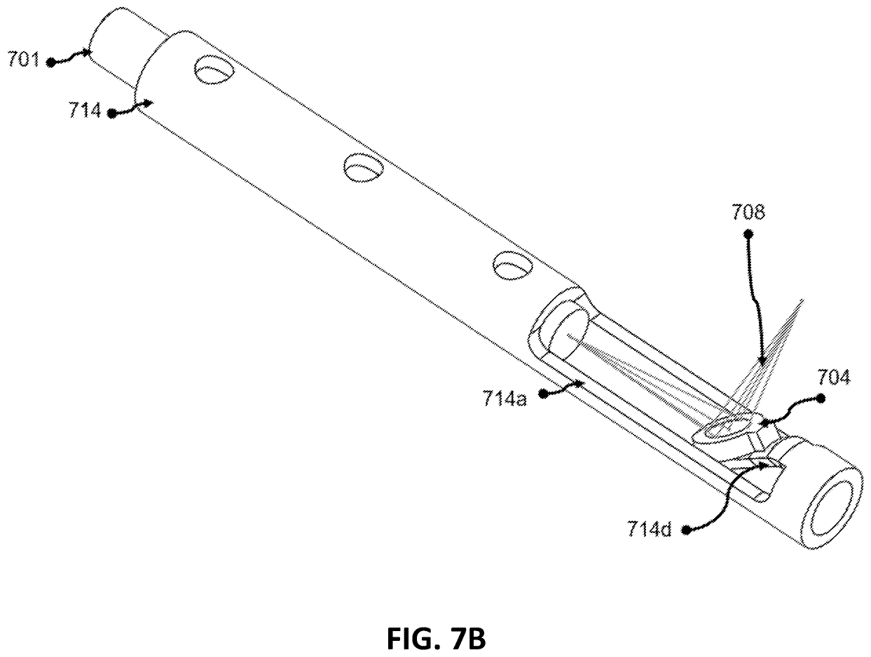

The structured slot and structured neck interface between the hypotube end wall and the lens provides a mechanical keying feature that facilitates reliable positioning during assembly. Unlike conventional press-fit or adhesive-only mounting approaches, the slot-neck interface helps establish a defined rotational orientation and a controlled mounting angle relative to the optical axis of the fiber prior to final bonding, reducing the need for active alignment equipment during manufacturing. Additionally, positioning of the lens mounting interface at the distal end wall of the hypotube, rather than along a sidewall, enables the lens optical axis to be oriented at a controlled angle relative to the fiber optical axis. This angular relationship can be designed to direct light toward specific anatomical targets during endoscopic procedures. The end wall mounting configuration also allows the lens to be positioned closer to the distal tip of the device, which can reduce the overall length of the imaging assembly and improve maneuverability within constrained luminal spaces. FIGS. 7A-9 show examples of such implementations.

FIGS. 7A and 7B illustrate hypotube-based lens mounting configurations in which the hypotube 714 serves as the lens mount, consolidating the functions of ferrule, mount, and alignment stage into a single structural element. In these configurations, the optical fiber 701 is coupled into the proximal end of the hypotube 714 and extends through the hypotube channel toward the distal end. The lens 704 is configured to direct light to and from the optical fiber 701 during imaging within the human body. The lens 704 is inserted into a window 714a formed on an end wall at the distal end of the hypotube 714 and secured in place. The window 714a comprises a structured slot on an outer perimeter of the end wall, and the lens 704 comprises a structured neck on an outer edge, the structured neck formed such that it passes through the structured slot in the end wall of the hypotube 714 when mounting the lens 704 in the hypotube 714. This slot-neck interface provides mechanical keying that facilitates repeatable angular positioning during assembly, establishing a defined rotational orientation and a controlled mounting angle relative to the optical axis of the fiber 701 prior to final bonding.

The lens 704 focuses the light rays 708 at a focal point 709, enabling imaging or illumination of target tissue within the human body. The hypotube 714 functions as both the lens holder and protector, providing mechanical support and shielding the optical components from the surrounding environment. In some configurations, the hypotube 714 includes one or more attachment windows 714b formed in a sidewall of the hypotube 714. These attachment windows 714b are configured to secure the optical fiber 701 into the hypotube 714, for example by applying an adhesive substance to the attachment windows 714b to bond the fiber 701 to the inner wall of the hypotube 714. The attachment windows 714b may also be used for adding adhesive to other components inside the hypotube 714, such as to secure a ferrule or other fiber-holding structure.

In some example configurations, a ferrule may be inserted into the hypotube 714 to facilitate handling and positioning of the optical fiber 701. The ferrule can contain a channel through which the fiber 701 is threaded, and the ferrule may be secured within the hypotube 714 by adhesive bonding or mechanical interference fit. The use of a ferrule can simplify fiber installation and provide additional mechanical stability, particularly in configurations where the fiber 701 must be precisely aligned with the lens 704 or other optical elements within the hypotube 714.

FIG. 7A shows an end cut on the hypotube to facilitate the lens mounting, according to an example embodiment. The hypotube 714 is used as the lens mount, with the optical fiber 701 coupled into the proximal end of the hypotube 714 and passing through the hypotube channel. The hypotube 714 includes a hypotube window 714a where optical components reside. The hypotube window 714a includes an installation window (also referred to as an end cut 714c) formed on the end wall at the distal end of the hypotube 714. The installation window comprises a structured slot on an outer perimeter of the end wall. The lens 704 comprises a structured neck on an outer edge that passes through the structured slot in the installation window during assembly.

The end cut 714c may be formed by laser cutting, mechanical cutting, or other precision machining techniques, and is configured to provide clearance for the structured neck of the lens 704 during insertion, allowing the lens 704 to be inserted through the installation window without interference from the hypotube sidewall. The structured neck may be separated from a carrier body used for handling the lens 704 either before insertion through the installation window or after the lens 704 is positioned within the hypotube window 714a, leaving the lens 704 mounted within the hypotube 714. The lens 704 focuses the light 708 emitted from the fiber 701 toward target tissue, and the hypotube 714 serves as both a lens holder and a protector simultaneously, providing mechanical support and environmental shielding for the optical components.

FIG. 7B shows a lens rest integrated into the hypotube, according to an example embodiment. In this configuration, the hypotube 719 functions as the lens mount, with the optical fiber 701 coupled into the proximal end of the hypotube 719 and passing through the hypotube channel. The hypotube 719 includes a hypotube window 719a where optical components reside. The hypotube window 719a includes an installation window (also referred to as an end cut) formed on the end wall at the distal end of the hypotube 719. The lens 704 is inserted into the hypotube window 719a through the installation window and securely positioned.

A lens rest 719d (e.g., a lens mount) is integrated into the hypotube 719 to securely hold the lens 704 in place. The lens rest 719d is formed from the hypotube body itself, for example by bending the sidewall of the hypotube 719 into the channel of the hypotube 719 to create a mounting surface for the lens 704. The lens rest 719d aids in the precise mounting of the lens 704 by providing a mechanical support surface that establishes the mounting angle of the lens 704 relative to the optical axis of the fiber 701.

The lens 704 may be mounted directly to the hypotube 719 by applying an adhesive substance to a distal side of the lens 704 within the hypotube window 719a. In some configurations, the adhesive substance is applied to the installation window on the end wall at the distal end of the hypotube 719, and capillary forces draw the adhesive substance to the distal side of the lens 704, improving bond uniformity and reducing the occurrence of voids or excess adhesive. The lens 704 focuses the light 708 emitted from the fiber 701, and the hypotube 719 provides both support and protection for the lens 704, consolidating multiple assembly functions into a single structural element.

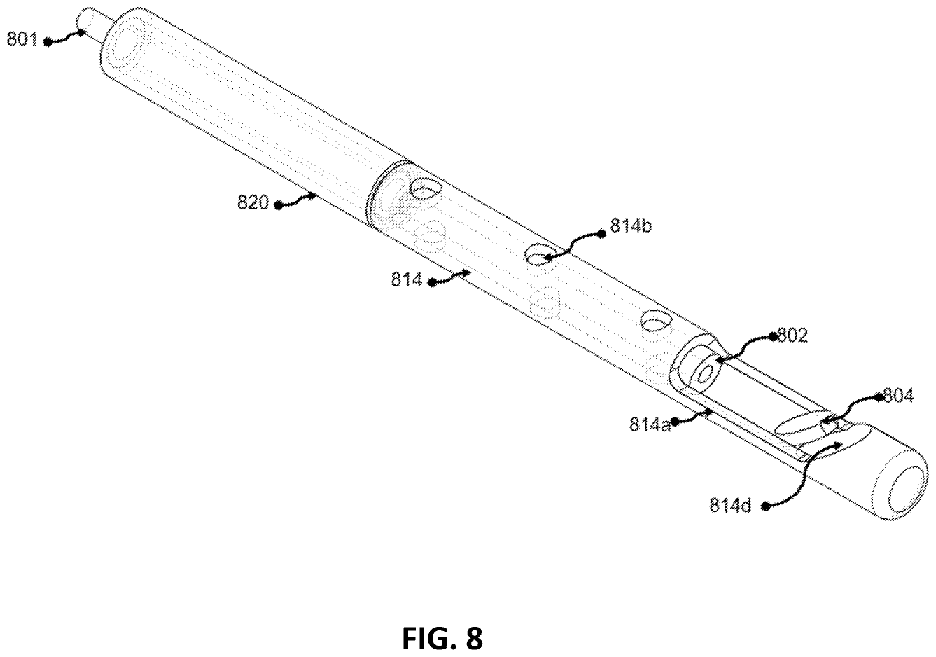

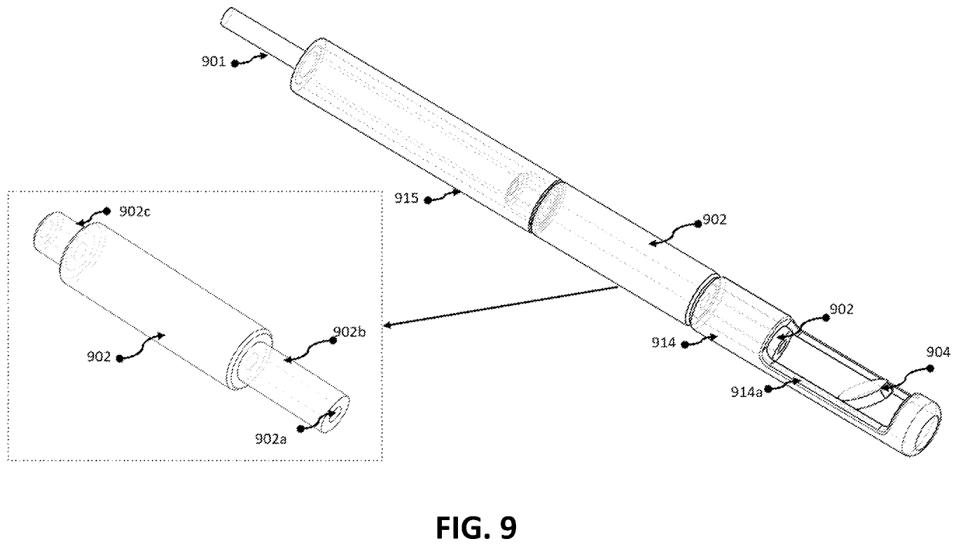

Hypotube lens mount configurations can include additional components that facilitate rotation of the optical assembly to enable circumferential imaging. The rotation allows the system to form a 360-degree image by rotating the lens 704 and fiber 701 about the longitudinal axis of the hypotube, enabling the imaging system to acquire cross-sectional images of tissue structures surrounding the endoscope. FIGS. 8 and 9 show such implementations, in which a torque coil is coupled to the proximal end of the hypotube to transmit rotational motion from an external motor or drive mechanism to the distal end of the imaging assembly.

FIG. 8 shows a hypotube lens mount configured to facilitate rotation, according to an example embodiment.

In the illustrated example, the proximal end of the hypotube 814 is attached to a torque coil 820 using an adhesive substance or welding. The torque coil 820 is used to transfer torque from a rotating source (such as a motor in an external imaging console) to the distal end of the imaging assembly, enabling the lens 804 and fiber 801 to rotate about the longitudinal axis of the hypotube 814. The torque coil 820 may have a length that varies from 10 mm to several meters, depending on the working length of the endoscopic device, and a diameter that ranges from 10 μm to 10 mm, depending on the size constraints of the application. During operation, the torque coil 820 rotates the hypotube 814, lens 804, and fiber 801 as a unit, enabling the imaging system to acquire 360-degree cross-sectional images of tissue structures.

In the illustrated example, the optical fiber 801 is threaded through the torque coil 820 and a ferrule 802, and the ferrule 802 is inserted into the hypotube 814. The hypotube 814 includes a hypotube window 814a where optical components reside. The hypotube window 814a includes an installation window (also referred to as an end cut) formed on the end wall at the distal end of the hypotube 814. The lens 804 is inserted into the hypotube window 814a through the installation window and onto a lens rest 814d of the hypotube 814, and is secured in place by applying an adhesive substance. The lens rest 814d is formed from the hypotube body and provides a mounting surface that establishes the mounting angle of the lens 804 relative to the optical axis of the fiber 801. The hypotube 814 functions as both the lens holder and protector, providing mechanical support and environmental shielding for the optical components. The hypotube 814 includes one or more attachment windows 814b formed in the sidewall, which can be used for adding adhesive to secure components inside the hypotube 814, such as the ferrule 802 or the fiber 801.

FIG. 9 shows a hypotube lens mount configured to facilitate rotation, according to an example embodiment.

In the illustrated example, the ferrule 902 has two extensions: a distal extension 902b and a proximal extension 902c. The distal extension 902b is configured to fit into the hypotube 914, and the proximal extension 902c is configured to fit into the torque coil 915. This dual-extension configuration provides mechanical coupling between the torque coil 915, the ferrule 902, and the hypotube 914, enabling rotational motion to be transmitted from the torque coil 915 through the ferrule 902 to the hypotube 914 and lens 904. The torque coil 915 is used to transfer torque from a rotating source to the distal end of the imaging assembly, and may have a length that varies from 10 mm to several meters and a diameter that ranges from 10 μm to 10 mm. During operation, the torque coil 915 rotates the ferrule 902, hypotube 914, lens 904, and fiber 901 as a unit, enabling the imaging system to acquire 360-degree cross-sectional images of tissue structures surrounding the endoscope.

In the illustrated configuration, the optical fiber 901 is threaded through the torque coil 915 and the ferrule 902, and the ferrule 902 is inserted into the hypotube 914 via the distal extension 902b. The hypotube 914 includes a hypotube window 914a where optical components reside. The hypotube window 914a includes an installation window (also referred to as an end cut) formed on the end wall at the distal end of the hypotube 914. The installation window comprises a structured slot on an outer perimeter of the end wall, and the lens 904 comprises a structured neck on an outer edge that passes through the structured slot during assembly. The lens 904 is inserted into the hypotube window 914a through the installation window and secured in place by applying an adhesive substance. The hypotube 914 functions as both the lens holder and protector, consolidating the functions of ferrule, mount, and alignment stage into a single structural element.

### Adhesive Substance Collection

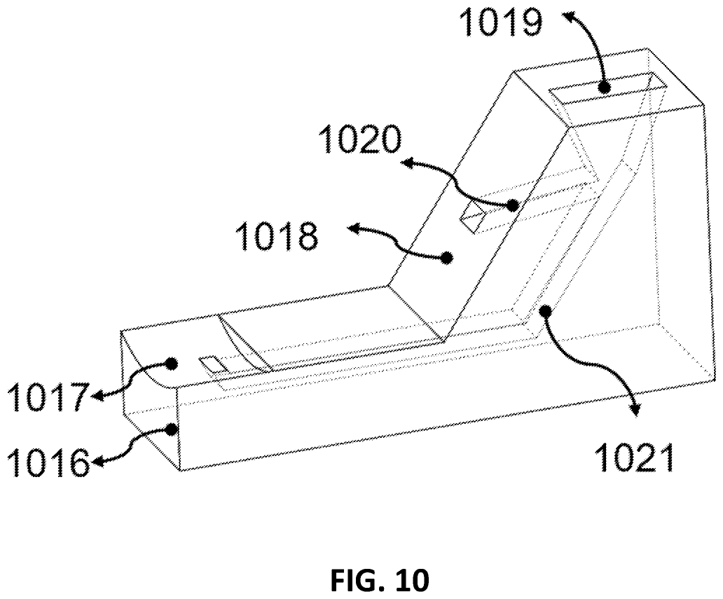

The system 100 includes various elements that allow for an adhesive substance (e.g., glue) to be dispensed to secure lenses. Due to the nature of adhesive substances, it is useful to also include elements that collect excess adhesive substance during assembly. FIGS. 10-12 illustrate various elements of the system for adhesive substance dispensing and collection.

FIG. 10 shows a mount with microchannels for adhesive substance dispensing, according to an example embodiment. The mount includes microchannels 1021 internal to the mount 1016 that enable adhesive substance to be dispensed in a controlled manner. This mount streamlines the assembly process by eliminating the need for precise manual adhesive substance application at specific points. The adhesive substance is dispensed at adhesive substance dispensing channel opening 1019 and guided through channel 1020 to the lens placement area 1018. In addition, channel 1021 guides the adhesive substance to the fiber/ferrule placement location 1017. Moreover, this approach minimizes the number of times that the adhesive substance needs to be applied manually (because the channel guides the adhesive substance from a single application to multiple points).

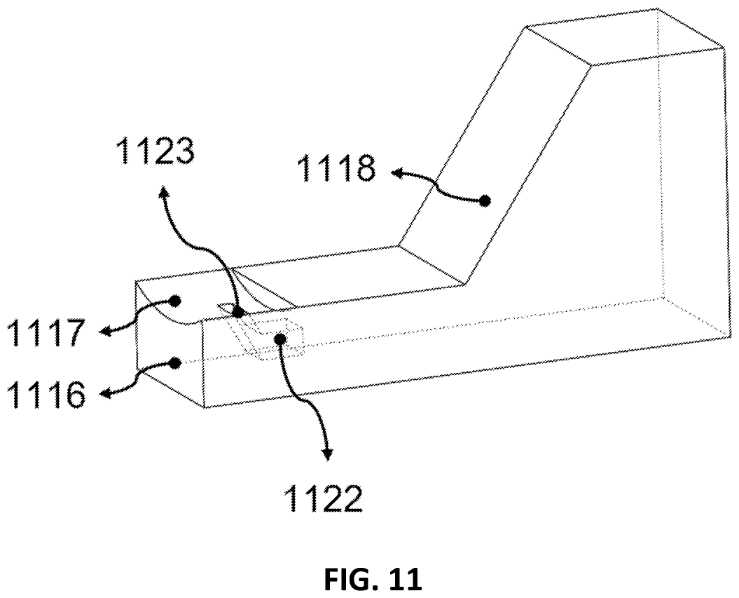

FIG. 11 shows a mount with an adhesive substance collecting reservoir, according to an example embodiment. Due to small footprint of elements if extra adhesive substance is applied it can block the elements e.g., fiber facet. This figure illustrates a mount 1116 with a reservoir 1122 to collect extra adhesive substance. Adhesive substance is applied to area 1117 and fiber/ferrule is placed in the same area. Extra adhesive substance will be collected through opening 1123 and stored in reservoir 1122. The lens will be placed in location 1118.

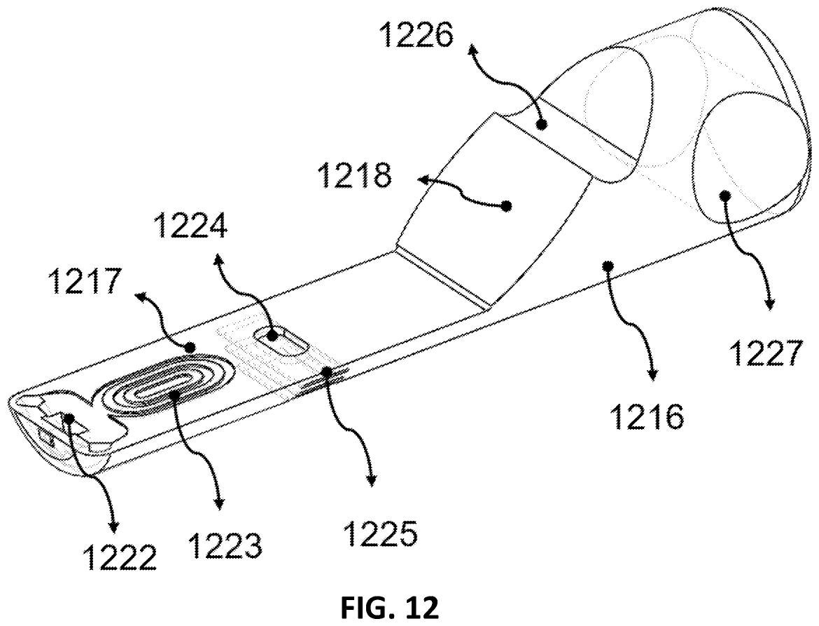

FIG. 12 shows a mount with an adhesive substance collecting reservoir, according to an example embodiment. Due to small footprint of elements if extra adhesive substance is applied it can block the elements e.g., fiber facet. In addition, due to small area, the connecting area to adhesive substance can be weak. To address this issue, fine channels can distribute and increase contact area. This figure illustrates a mount 1216 with microchannels 1223 to collect extra adhesive substance and increase adhesion. Adhesive substance is applied to microchannels 1223. The adhesive substance is well distributed in this area and extra adhesive substance would be collected by reservoir 1222. After fiber/ferrule is placed over area 1217 extra adhesive substance can flow towards opening 1224 which can facilitate extra adhesive substance collection from distal end into reservoir 1225. The lens is placed on a surface 1218. The extra adhesive substance applied to lens area can be collected by notch 1226. Due to the small mount size a through hole 1227 is placed in the mount to facilitate handling. The mount diameter can vary from 10 μm to 5 mm with typical size of 300 μm. The length can range from 100 μm to 10 mm with typical size of 2 mm.

In various configurations, the adhesive substance used in the assembly processes described herein may comprise various materials suitable for bonding optical components within miniaturized endoscopic systems. In some examples, the adhesive substance may be an epoxy resin, such as a two-part epoxy that cures upon mixing or a UV-curable epoxy that solidifies upon exposure to ultraviolet light. In other configurations, the adhesive substance may be a cyanoacrylate adhesive (commonly referred to as super glue), which provides rapid bonding and is suitable for applications where fast assembly is desired. The adhesive substance may also be a silicone-based adhesive, which offers flexibility and resistance to temperature variations, or an acrylic adhesive, which provides strong bonding and optical clarity. In some cases, the adhesive substance may be a thermoplastic adhesive that softens upon heating and solidifies upon cooling, enabling rework or repositioning of components during assembly. The selection of adhesive substance depends on factors such as curing time, bond strength, optical transparency, thermal stability, and compatibility with the materials being bonded, including the lens substrates, hypotube materials, and optical fiber coatings.

Additionally, the adhesive substance dispensing and collection mechanisms described above represent example configurations applicable to lens mounts, and similar principles may be extended to other components of the endoscopic imaging system. For instance, microchannels for controlled adhesive substance dispensing may be incorporated into ferrule mounts to facilitate bonding of the optical fiber within the ferrule channel, reducing the need for precise manual application and improving bond uniformity.

To illustrate, in hypotube-based assemblies, adhesive substance collection reservoirs may be integrated into the hypotube sidewall or end wall to capture excess adhesive substance during lens installation, preventing obstruction of the optical path or interference with the imaging window. In prism-based configurations, adhesive substance dispensing channels may be formed in the outer sheath to guide adhesive substance to the prism mounting interface, enabling secure attachment of the prism within the sheath while minimizing manual intervention. Additionally, adhesive substance collection features may be incorporated into mounts that secure multiple optical elements in cascaded configurations, where excess adhesive substance from one bonding operation could otherwise migrate to adjacent components and degrade optical performance.

Finally, the adhesive substance management techniques disclosed herein are not limited to the specific mount geometries illustrated in FIGS. 10-12, and may be adapted to various structural configurations and assembly sequences depending on the specific requirements of the endoscopic imaging system.

### Optical Shielding and Alignment

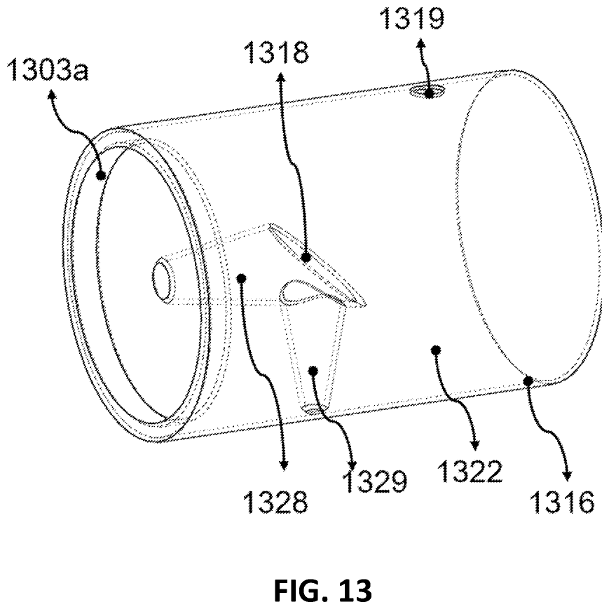

FIG. 13 shows a mount with optical shielding, according to an example embodiment. Due to the small size of the system, the imaging assembly can be sensitive to stray light that may enter through gaps between components or reflect from internal surfaces, potentially degrading image quality and introducing noise artifacts. The mount 1316 illustrated in FIG. 13 addresses this challenge by incorporating features that enable the dispensing and containment of an optical shielding material, such as a black epoxy, carbon-loaded polymer, or other light-absorbing substance.

The shielding material may be dispensed into opening 1319 such that it flows into reservoir 1322, where it solidifies to form a light-blocking barrier around the optical path. The fiber or ferrule is inserted into 1303a, and light emitted from the fiber travels through channel 132 while being shielded from stray light by the surrounding shielding material. The lens is positioned at 1318, and focused light passes through channel 1329, which is similarly shielded to prevent unwanted light from entering or exiting the optical path. This shielding configuration reduces optical noise, improves signal-to-noise ratio, and enhances the overall imaging performance of the endoscopic system by minimizing interference from ambient light or reflections from non-optical surfaces within the device.

Additionally, the endoscopic imaging systems disclosed herein incorporate features that facilitate target alignment of optical components, particularly with respect to the angular positioning of lenses relative to the optical axis of the fiber. In many configurations, the lens is mounted within the hypotube or other mounting structure at a controlled mounting angle that directs light toward specific anatomical targets during endoscopic procedures. This mounting angle is established by the mechanical interface between the lens and the mount, such as the structured slot and structured neck interface described above, or by the geometry of a lens rest formed in the hypotube sidewall.

Within this context, the target angular position includes both a rotational component around the optical axis and a tilt angle of the lens relative to the optical axis. The rotational component determines the circumferential orientation of the lens around the fiber, while the tilt angle controls the inclination of the lens surface relative to the optical axis (thereby directing light radially outward toward tissue structures surrounding the endoscope). By designing the mechanical features to provide a target angular position in both rotational and tilt dimensions, the system enables passive alignment of the lens during assembly, reducing the need for active alignment equipment and improving manufacturing repeatability.

However, due to manufacturing tolerances in the lens geometry, hypotube dimensions, and assembly process, the actual mounting angle of the lens may vary within a range defined by a minimum angle and a maximum angle. These minimum and maximum angles are determined by the absolute and relative positions of the lens, the optical fiber, and the hypotube, as well as by the dimensional tolerances of the structured slot, structured neck, lens rest, or other alignment features. For example, if the structured neck of the lens is slightly thicker or thinner than the nominal dimension, or if the structured slot in the hypotube end wall is slightly wider or narrower than designed, the resulting mounting angle may deviate from the target angular position. Similarly, variations in the position of the lens rest relative to the optical axis of the fiber can introduce angular deviations in the mounted lens.

The endoscopic imaging system is configured to compensate for variations in the mounting angle between the minimum angle and the maximum angle, enabling the system to maintain acceptable optical performance despite these manufacturing tolerances. Compensation may be achieved through various mechanisms, including optical design strategies that provide sufficient depth of focus or field of view to accommodate angular deviations, or through post-processing of imaging data to correct for known or measured angular offsets. In some configurations, the imaging console or associated software may apply geometric transformations to the acquired images to account for the mounting angle of the lens, enabling accurate reconstruction of tissue structures even when the lens is not positioned at the exact target angular position. By designing the system to operate within a defined range of mounting angles, the disclosed configurations reduce the sensitivity of optical performance to manufacturing variations, improving yield and reducing the need for tight tolerances or active alignment during assembly.

### Additional Considerations

In the description above, for purposes of explanation, numerous specific details are set forth in order to provide a thorough understanding of the illustrated system and its operations. It will be apparent, however, to one skilled in the art that the system can be operated without these specific details. In other instances, structures and devices are shown in block diagram form in order to avoid obscuring the system.

Reference in the specification to “one embodiment” or “an embodiment” means that a particular feature, structure, or characteristic described in connection with the embodiment is included in at least one embodiment of the system. The appearances of the phrase “in one embodiment” in various places in the specification are not necessarily all referring to the same embodiment.

Some portions of the detailed descriptions are presented in terms of algorithms or models and symbolic representations of operations on data bits within a computer memory. An algorithm is here, and generally, conceived to be steps leading to a desired result. The steps are those requiring physical transformations or manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Some of the operations described herein are performed by a computer physically mounted within a machine. This computer may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), random access memories (RAMs), EPROMs, EEPROMs, magnetic or optical cards, or any type of non-transitory computer readable storage medium suitable for storing electronic instructions.

The figures and the description above relate to various embodiments by way of illustration only. It should be noted that from the following discussion, alternative embodiments of the structures and methods disclosed herein will be readily recognized as viable alternatives that may be employed without departing from the principles of what is claimed.

One or more embodiments have been described above, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct physical or electrical contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B is true (or present).

In addition, use of the “a” or “an” are employed to describe elements and components of the embodiments herein. This is done merely for convenience and to give a general sense of the system. This description should be read to include one or at least one and the singular also includes the plural unless it is obvious that it is meant otherwise.

Upon reading this disclosure, those of skill in the art will appreciate still additional alternative structural and functional designs for a system and a process for operating a vehicle in an environment with moisture including a control system executing a semantic segmentation model. Thus, while particular embodiments and applications have been illustrated and described, it is to be understood that the disclosed embodiments are not limited to the precise construction and components disclosed herein. Various modifications, changes and variations, which will be apparent to those, skilled in the art, may be made in the arrangement, operation and details of the method and apparatus disclosed herein without departing from the spirit and scope defined in the appended claims.

What is claimed is:

1. An endoscopic imaging system comprising:
an optical fiber configured for translating light during imaging within a human body;
a hypotube comprising:
a proximal end and a distal end, wherein the optical fiber is coupled into the proximal end of the hypotube; and
a window formed on an end wall at the distal end of the hypotube, the window comprising a structured slot on an outer perimeter of the end wall; and
a lens configured to direct light to and from the optical fiber during imaging, the lens comprising a structured projection on an outer edge, the structured projection formed such that it passes through the structured slot in the end wall of the hypotube when mounting the lens in the hypotube.

2. The endoscopic imaging system of claim 1, wherein the hypotube comprises one or more vision windows, each of the one or more vision windows configured for transmitting light reflected off (or transmitted through) the lens or collecting light reflected from surfaces in the human body.

3. The endoscopic imaging system of claim 2, wherein the window formed on the end wall is not one of the one or more vision windows.

4. The endoscopic imaging system of claim 1, wherein the hypotube additionally comprises:
one or more attachment windows in a sidewall of the hypotube, the one or more attachment windows configured to secure the optical fiber into the hypotube, and
wherein the optical fiber is secured into the hypotube by applying an adhesive substance to the one or more attachment windows.

5. The endoscopic imaging system of claim 1, wherein:
the lens is directly mounted to the hypotube by applying an adhesive substance to a distal side of the lens within the hypotube, and
the adhesive substance is applied to the window on the end wall at the distal end of the hypotube and capillary forces draw the adhesive substance to the distal side of the lens.

6. The endoscopic imaging system of claim 1, wherein the lens is mounted to the hypotube at a mounting angle relative to a light axis of the endoscopic imaging system, the mounting angle between a minimum angle and a maximum angle (e.g., between 0 and 180 degrees).

7. The endoscopic imaging system of claim 6, wherein the minimum angle and the maximum angle is determined by absolute and relative positions of at least the lens, the optical fiber, and the hypotube.

8. The endoscopic imaging system of claim 6, wherein the imaging system is configured to compensate for variations in the mounting angle between the minimum angle and the maximum angle.

9. The endoscopic imaging system of claim 1, further comprising:
a lens rest, wherein the lens is mounted on a surface of the lens rest within the hypotube, and wherein the lens reflects or transmits incoming light from the optical fiber.

10. The endoscopic imaging system of claim 9, wherein:
the lens rest is formed from a sidewall of the hypotube and is formed by bending the sidewall of the hypotube into a channel of the hypotube.

11. The endoscopic imaging system of claim 9, wherein the lens rest comprises:
an insert for the hypotube, wherein the insert is configured for inserting into the distal end of the hypotube such that the lens is rested on a proximal surface of the insert.

12. The endoscopic imaging system of claim 11, wherein the insert comprises one or more channels, each of the one or more channels configured to receive an adhesive substance on a distal side of the insert to translate the adhesive substance to a proximal side of the insert.

13. The endoscopic imaging system of claim 1, wherein the lens comprises a proximal side and a distal side, the proximal side configured for reflecting or transmitting light in the endoscopic imaging system, and the distal side configured for resting the lens within the endoscopic imaging system.

14. The endoscopic imaging system of claim 1, further comprising:
a self-aligning lens mount, the self-aligning lens mount comprising one or more attachment structures configured to attach the lens to the self-aligning lens mount.

15. The endoscopic imaging system of claim 14, wherein the one or more attachment structures are positioned such that the one or more attachment structures clamp the lens to the self-aligning lens mount along an outer perimeter of the lens.

16. The endoscopic imaging system of claim 14, wherein the one or more attachment structures exert a spring force on a surface of the lens to couple the lens to the self-aligning mount.

17. The endoscopic imaging system of claim 1, further comprising:
a prism mount, wherein:
the lens is coupled to the prism mount, and
the prism mount directs light to and from the lens during imaging.

18. The endoscopic imaging system of claim 17, wherein the prism mount directs light to and from the lens during imaging through a body of the prism mount.

19. The endoscopic imaging system of claim 17, further comprising:
an additional lens configured to direct light to and from the optical fiber during imaging,
wherein:
the additional lens is coupled to the prism mount, and
the prism mount directs light to and from the lens during imaging.

20. The endoscopic imaging system of claim 1, further comprising:
one or more adhesive substance channels, each of the one or more adhesive substance channels formed to translate an adhesive substance from a first area of the endoscopic imaging system to a second area of the endoscopic imaging system,
wherein capillary forces translate the adhesive substance from the first area to the second area through the one or more adhesive substance channels.
